(19) United States
(12) Patent Application Publication
(10) Pub. No.: US 2010/0256991 A1
(43) Pub. Date: Oct. 7, 2010

(54) MEDICAL DIAGNOSIS SUPPORT APPARATUS

(75) Inventors: Ryo Ishikawa, Kawasaki-shi (JP); Akihiro Katayama, Yokohama-shi (JP); Yoshio Iizuka, Yokohama-shi (JP); Kazuhiro Miyasa, Kawasaki-shi (JP)

Correspondence Address: CANON U.S.A. INC. INTELLECTUAL PROPERTY DIVISION, 15975 ALTON PARKWAY, IRVINE, CA, 92618-3731, US

(73) Assignee: CANON KABUSHIKI KAISHA, Tokyo (JP)

(21) Appl. No.: 12/680,537

(22) PCT Filed: Sep. 26, 2008
(86) PCT No.: PCT/JP2008/067443

§ 371 Date: Mar. 26, 2010

(30) Foreign Application Priority Data
Sep. 27, 2007 (JP) 2007-252378

(51) Int. Cl. A61B 5/00

(52) U.S. Cl. 705/3

(57) ABSTRACT

A medical diagnosis support apparatus analyzes the interpretation report information created by a user, generates analysis result information in which each medical information extracted from the interpretation report information is associated with an item, and detects a difference for each item by comparing the diagnosis support information, in which each medical information obtained by computer processing of medical examination data is associated with an item, with the generated analysis result information. The apparatus determines an importance concerning a difference for each detected item. The apparatus then presents the detected difference, together with display of the contents of the interpretation report information, while changing the display form in accordance with the determined importance.

### TECHNICAL FIELD

The present invention relates to a medical diagnosis support system which can be used to improve the diagnosis efficiency of medical examination data by a doctor by comparing and contrasting a plurality of pieces of diagnosis support information such as doctor's findings and analyses by a computer about medical examination data such as medical images.

### BACKGROUND ART

In the medical field, the digitization of medical images obtained by capturing patient images has been implemented. Medical image data of this type are generated by, for example,
CR (Computed Radiography) apparatus,
CT (Computed Tomography) apparatus,
MRI (Magnetic Resonance Imaging) apparatus, and
ultrasound apparatus (US: Ultrasound System). At the time of diagnosis, generated medical image data is displayed on a monitor. A doctor as a user interprets the medical image displayed on the monitor and observes the state of a lesion or changes with time.

For reducing the load of such interpretation on a doctor, a medical image processing apparatus called a computer-aided diagnosis apparatus (CAD: Computer-Aided Diagnosis) has been developed. The medical image processing apparatus automatically detects a lesion as an abnormal shadow candidate by performing image analysis on the above medical image data. This abnormal shadow detection processing is the processing of detecting, using a computer, an abnormal shadow candidate such as an abnormal tumor shadow indicating a cancer or the like or a high-density minute calcification shadow on the basis of image data representing radiograms. This can reduce the load of interpretation on a doctor and improve the accuracy of an interpretation result.

When such a CAD is to be used in an actual clinical case, a doctor interprets radiograms first, and then refers to the diagnosis support information output from the CAD to compare it with the interpretation result. In this operation, more specifically, the doctor associates finding information on an interpretation report as an interpretation result, which the doctor has written by himself/herself, with finding information of the diagnosis support information calculated by the CAD to find an oversight, a detection error, a difference in finding, and the like. In this case, it is possible to reduce the operation load on the doctor by automatically comparing the interpretation report created by the doctor with the diagnosis support information calculated by the CAD and presenting differences (patent reference 1).

Patent reference 1: Japanese Patent No. 3085724

### DISCLOSURE OF INVENTION

### Problems that the Invention is to Solve

The differences between the interpretation report and the diagnosis support information calculated by the CAD include medically important differences concerning diagnosis and medically unimportant information such as expressions and detailedness of description on the interpretation report. According to patent reference 1, since there is no consideration to such differences in medical importance, even a detection result on a medically unimportant difference is presented to the doctor. This makes the doctor feel troublesome. In addition, medically important differences may be mixed in other information. This may disable to effectively present the diagnosis support information obtained by the CAD to the doctor.

The present invention has been made in consideration of the above problem, and has as its object to improve the efficiency of interpreting operation by a doctor or the like by providing display suitable for a comparison result on the interpretation result obtained by the doctor or the like as a user and the diagnosis support information obtained by a CAD.

### Means of Solving the Problems

In order to solve the above problems, a diagnosis support apparatus according to an aspect of the present invention has the following arrangement. That is,

there is provided a medical diagnosis support apparatus which supports diagnosis by a doctor based on medical examination data, comprising analysis means for analyzing interpretation report information created by a user and generating analysis result information in which each medical information extracted from the interpretation report information is associated with an item, detection means for detecting a difference for each item by comparing diagnosis support information, in which each medical information obtained by computer processing of medical examination data is associated with an item, with the analysis result information obtained by the analysis means, determination means for determining an importance concerning a difference for each item detected by the detection means, and presentation means for presenting the difference detected by the detection means, together with display of contents of the interpretation report information, while changing a display form in accordance with the importance determined by the determination means.

In addition, according to another aspect of the present invention, there is provided a control method for a medical diagnosis support apparatus which supports diagnosis by a doctor based on medical examination data, comprising an analysis step of analyzing interpretation report information created by a user and generating analysis result information in which each medical information extracted from the interpretation report information is associated with an item, a detection step of detecting a difference for each item by comparing diagnosis support information, in which each medical information obtained by computer processing of medical examination data is associated with an item, with the analysis result information obtained in the analysis step, a determination step of determining an importance concerning a difference for each item detected in the detection step, and a presentation step of presenting the difference detected in the detection step, together with display of contents of the interpretation report information, while changing a display form in accordance with the importance determined in the determination step.

### Effects of the Invention

The present invention can provide display suitable for a comparison result on the interpretation result obtained by a doctor or the like as a user and the diagnosis support information obtained by a CAD and improve the efficiency of interpreting operation by the doctor or the like.

Other features and advantages of the present invention will be apparent from the following description taken in conjunction with the accompanying drawings, in which like reference characters designate the same or similar parts throughout the figures thereof.

### BRIEF DESCRIPTION OF DRAWINGS

The accompanying drawings, which are incorporated in and constitute a part of the specification, illustrate embodiments of the invention and, together with the description, serve to explain the principles of the invention.

FIG. 1 is a block diagram showing an example of the configuration of a medical diagnosis support apparatus according to the first embodiment;

FIG. 2 is a functional block diagram showing an example of the functional arrangement of a medical diagnosis support apparatus 1 according to the first embodiment;

FIG. 3 is a flowchart showing a processing sequence in the medical diagnosis support apparatus according to the first embodiment;

FIG. 4 is a view for explaining processing in an interpretation report analysis unit 11;

FIG. 5 is a view for explaining processing in a difference detection unit 14;

FIG. 6 is a view for explaining processing in the difference detection unit 14;

FIG. 7 is a view for explaining processing in the difference detection unit 14;

FIG. 8 is a view showing an example of the data arrangement of importance rank information in a term importance dictionary 150; and

FIG. 9 is a view showing a display example of a processing result by a difference presenting unit.

### BEST MODE FOR CARRYING OUT THE INVENTION

Preferred embodiments of a medical diagnosis support apparatus and method according to the present invention will be described in detail below with reference to the accompanying drawings. The scope of the present invention is not limited to the following embodiments.

### First Embodiment

{Hardware Arrangement}

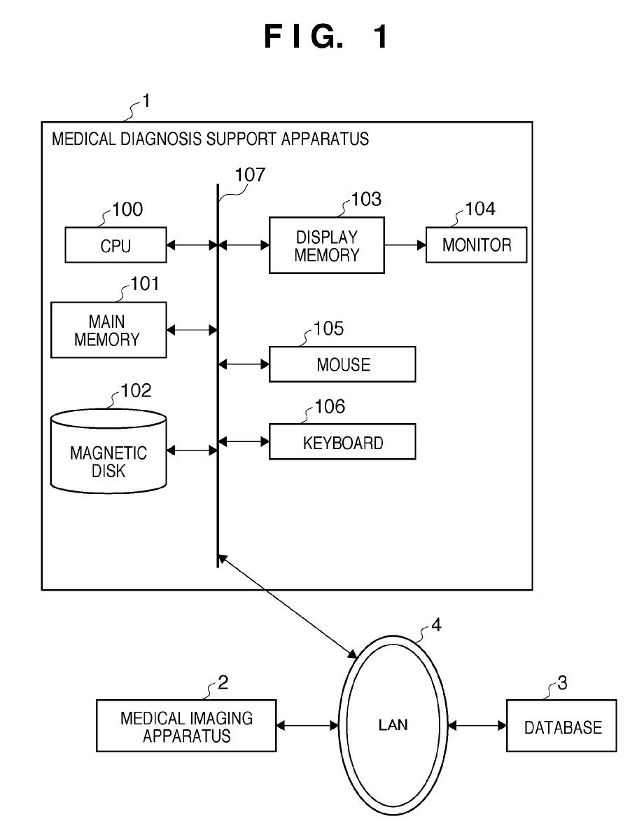

The arrangement of a medical diagnosis support apparatus according to the first embodiment which supports diagnosis by a doctor based on medical examination data. FIG. 1 is a block diagram showing an example of the arrangement of equipment according to the first embodiment. A medical diagnosis support apparatus 1 in FIG. 1 includes a central processing unit (CPU) 100, a main memory 101, a magnetic disk 102, a display memory 103, a monitor 104, a mouse 105, and a keyboard 106.

As shown in FIG. 1, the medical diagnosis support apparatus 1 can be connected to a medical imaging apparatus 2, for example, an X-ray CT apparatus, MR apparatus, US apparatus, X-ray apparatus, or nuclear medicine apparatus, which can capture an image of an object to be examined. The medical diagnosis support apparatus 1 may be connected to a database 3 which stores the medical images captured by the medical imaging apparatus 2 and medical examination data including information necessary for diagnosis support processing. As shown in FIG. 1, these apparatuses can be connected to each other via a network such as a local area network (LAN) 4 or can be directly connected to each other via dedicated interfaces.

The CPU 100 mainly controls the operation of each constituent element of the medical diagnosis support apparatus 1. The main memory 101 stores control programs to be executed by the CPU 100 or provides a work area when the CPU 100 executes a program. The magnetic disk 102 stores an operating system (OS), device drives for peripheral devices, various kinds of application software including programs for executing, for example, diagnosis support processing (to be described later). The display memory 103 temporarily stores display data for the monitor 104. The monitor 104 is, for example, a CRT monitor or a liquid crystal monitor, and displays images based on data from the display memory 103. The mouse 105 and the keyboard 106 are operated by the user to perform pointing input operation and input characters and the like. The respective constituent elements described above are connected to each other via a common bus 107.

Assume that in this embodiment, the medical diagnosis support apparatus 1 reads out medical images and the like from the database 3 via the LAN 4. However, the present invention is not limited to this. For example, a storage device such as an FDD, CD-RW drive, MO drive, and ZIP drive can be connected to the medical diagnosis support apparatus 1 to allow it to read medical images and the like from the drives. In addition, the medical diagnosis support apparatus 1 can directly acquire medical images and the like from the medical imaging apparatus 2 via the LAN 4.

{Functional Arrangement}

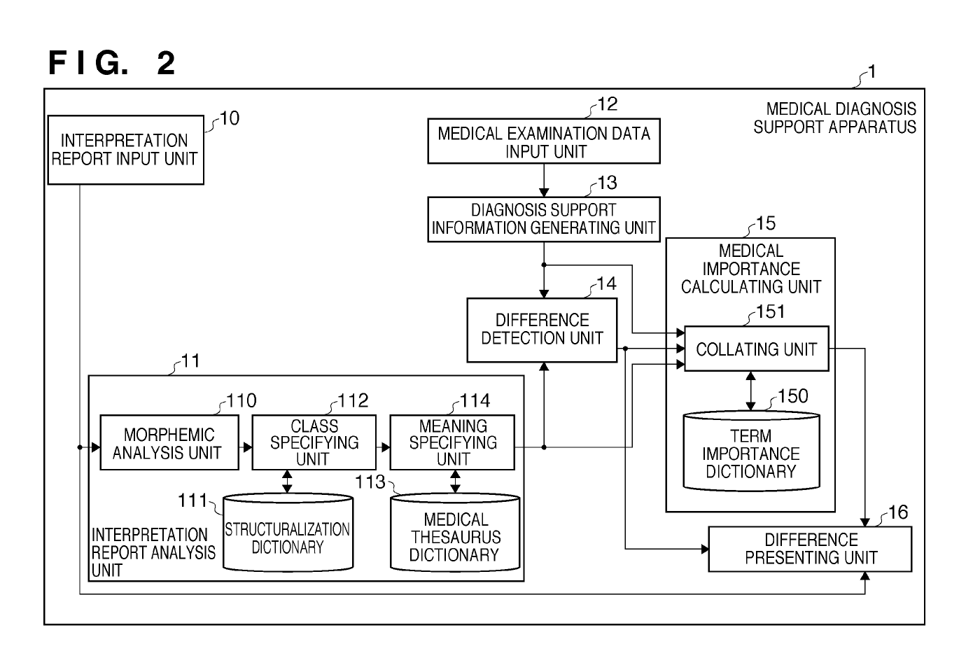

The functional arrangement of the medical diagnosis support apparatus 1 according to this embodiment will be described next with reference to FIG. 2. FIG. 2 is a functional block diagram of the medical diagnosis support apparatus 1 in this embodiment. As shown in FIG. 2, the medical diagnosis support apparatus 1 includes an interpretation report input unit 10, an interpretation report analysis unit 11, a medical examination data input unit 12, a diagnosis support information generating unit 13, a difference detection unit 14, a medical importance calculating unit 15, and a difference presenting unit 16. The above units are implemented by causing the CPU 100 of the medical diagnosis support apparatus 1 to execute the computer programs stored in the main memory 101 and cooperate with the hardware of the medical diagnosis support apparatus 1. Each functional unit will be described below.

<Interpretation Report Input Unit 10>

The interpretation report input unit 10 inputs interpretation report information as a result of interpretation by a doctor as a user to the medical diagnosis support apparatus 1 on the basis of medical examination data.

Input via Storage Medium/Network

For example, the interpretation report input unit 10 can be configured to receive, via a network, data from a database on which the interpretation report information created by the doctor who has interpreted radiograms is recorded. The interpretation report input unit 10 can also be configured to read out data from storage devices, for example, various kinds of storage media including an FDD, CD-RW drive, MO drive, and ZIP drive, connected to the medical diagnosis support apparatus 1.

Computer Free Sentence Input

The interpretation report input unit 10 can also be configured to allow the doctor who has interpreted radiograms to directly input interpretation report information as an interpretation result to the computer by using a keyboard, a mouse, and the like.

Fixed Format

In addition, the interpretation report input unit 10 can be configured to allow the doctor who has interpreted radiograms to create an interpretation report by preparing, in advance, a fixed format and choices of information to be entered in the respective items in the format and making the doctor select information from choices.

OCR (Optical Character Recognition)

The interpretation report input unit 10 may be configured such that a paper surface reader such as a paper scanner is connected to an optical character recognition apparatus. According to this arrangement, the scanner reads the sentences handwritten by the doctor who has interpreted radiograms, and the computer creates interpretation report information in an editable form by performing OCR processing.

<Interpretation Report Analysis Unit 11>

The interpretation report analysis unit 11 analyzes interpretation repot information as an interpretation result input by the doctor using the interpretation report input unit 10, thereby analyzing the medical meaning of the information.

For example, as shown FIG. 2, the interpretation report analysis unit 11 includes a morphemic analysis unit 110, a class specifying unit 112, and a meaning specifying unit 114, and holds a structuralization dictionary 111 and a medical thesaurus dictionary 113.

First of all, the morphemic analysis unit 110 segments interpretation report information written in natural sentences, input by the interpretation report input unit 10, into words (morphemes). The structuralization dictionary 111 is a dictionary in which words concerning medical care and the corresponding classes are registered. The types of classes to be registered in the structuralization dictionary 111 are not limited. For example, “region”, “symptom”, “name of disease”, and the like can be registered in the structuralization dictionary 111, as needed. The class specifying unit 112 specifies the class of each word obtained by the morphemic analysis unit 110 by referring to the structuralization dictionary 111, thereby generating structuralized data of interpretation report information. Structuralized data is data which is structuralized and includes elements having attributes associated with medical care, for example, “region”, “symptom”, and “name of disease”.

In the medical thesaurus dictionary 113, words are systematized based on a conceptual hierarchical relationship by an ontology which describes information indicating the relationship between words associated with medical care. That is, in the medical thesaurus dictionary 113, words are hierarchically arranged such that conceptually high-level words (superordinate words) are located at higher levels in a hierarchical structure, and conceptually low-level words (subordinate words) are located at lower levels in the hierarchical structure. The meaning specifying unit 114 determines a thesaurus code expressing the hierarchical position of the content (word) of an element having an attribute called “region” contained in the structuralized data of the interpretation report by referring to the medical thesaurus dictionary 113. The contents of concrete processing using these structures will be described in detail later.

In another arrangement in which the interpretation report input unit 10 selects and inputs words from choices, morphemic analysis need not be performed. In this case, the interpretation report analysis unit 11 can also be formed by connecting a thesaurus dictionary associated with words prepared as choices in advance in the interpretation report input unit 10 to a means for collation with the dictionary.

<Medical Examination Data Input Unit 12>

The medical examination data input unit 12 inputs medical examination data to the medical diagnosis support apparatus 1.

Example of Connection to LAN, CD-ROM, or the Like

The medical examination data input unit 12 can receive data from a database storing captured medical image data via a LAN. The medical examination data input unit 12 may also be configured to read medical image data from storage devices connected to the medical diagnosis support apparatus 1, for example, various kinds of storage media such as an FDD, CD-RW drive, MO drive, and ZIP drive.

Example of Connection to Imaging Device

The medical examination data input unit 12 may also be configured to be connected to a medical imaging apparatus, for example, an X-ray CT apparatus, MR apparatus, US apparatus, X-ray apparatus, or a nuclear medicine apparatus, which can capture an image of an object to be examined, and to directly input medical image data from these devices.

Example of Handling Also Medical Record Information

The medical examination data input unit 12 may be configured to input past and current interpretation reports and medical examination data including information necessary for diagnosis support processing as well as medical image data. In this case, the medical examination data input unit 12 may be configured to allow the user to directly input these data or to read out data from various kinds of storage media such as an FDD, CD-RW drive, MO drive, and ZIP drive, on which information is recorded. In addition, the medical examination data input unit 12 may be configured to be connected, via a LAN, to a database on which these data are recorded, and receive data from it.

<Diagnosis Support Information Generating Unit 13>

The diagnosis support information generating unit 13 generates medical diagnosis support information concerning an object to be examined, based on the medical examination data input from the medical examination data input unit 12. The contents of concrete processing associated with the diagnosis support information generating unit 13 will be described in detail later.

<Difference Detection Unit 14>

The difference detection unit 14 detects the difference between the medical meaning of the interpretation result obtained by the doctor, which is extracted by analysis by the interpretation report analysis unit 11, and the diagnosis support information generated by the diagnosis support information generating unit 13.

<Medical Importance Calculating Unit 15>

The medical importance calculating unit 15 calculates the medical importance of the difference between the interpretation result obtained by the doctor, which is detected by the difference detection unit 14, and diagnosis support information.

For example, as shown in FIG. 2, the medical importance calculating unit 15 includes the term importance dictionary 150 and a collating unit 151, and can obtain a medical importance in accordance with the content of the difference detected by the difference detection unit 14. The medical importance calculating unit 15 can be configured to be connected to the interpretation report analysis unit 11 and the diagnosis support information generating unit 13 so as to calculate a medical importance, also based on the processing results obtained by them. The term importance dictionary 150 has data defining the medical importances of the words written on an interpretation report. The collating unit 151 collates/searches the term importance dictionary 150 for a word matching the difference information detected by the difference detection unit 14, and determines its importance. The contents of concrete processing using these functional arrangements will be described in detail later.

<Difference Presenting Unit 16>

The difference presenting unit 16 presents, on the monitor 104, the difference information detected by the difference detection unit 14 on the interpretation result obtained by the doctor, which is input by the interpretation report input unit 10, upon changing the display form in accordance with the medical importance calculated by the medical importance calculating unit 15.

{Description of Processing}

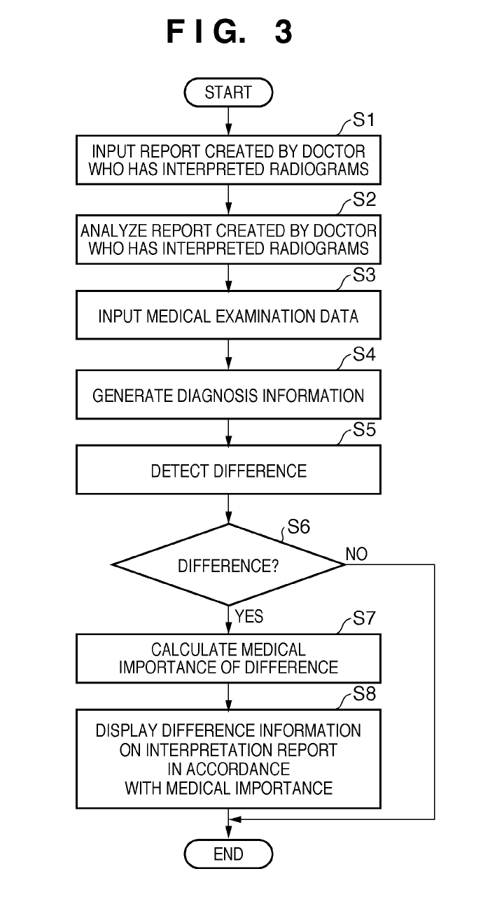

A concrete processing sequence executed by the medical diagnosis support apparatus 1 of this embodiment will be described next with reference to the flowchart of FIG. 3.

(Step S1)

The interpretation report input unit 10 inputs the interpretation report (interpretation report information) written by the doctor who has interpreted radiograms.

(Step S2)

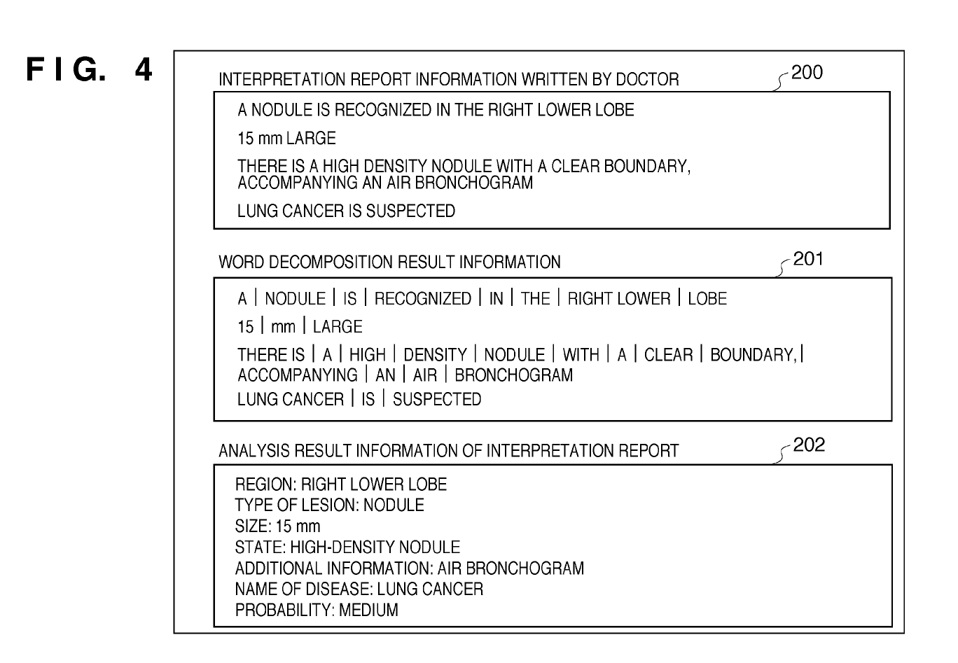

The interpretation report analysis unit 11 analyzes the interpretation report information input in step S1 and extracts a medical meaning contained in the interpretation report information. A concrete example of the processing in step S2 will be described with reference to FIG. 4. Referring to FIG. 4, interpretation report information 200 written by the doctor is an example of the interpretation report information input in step S1. In this case, the interpretation report information is written as free sentences by the doctor. First of all, the interpretation report analysis unit 11 decomposes the interpretation report information 200 written by the doctor on a word basis by morphemic analysis to obtain word decomposition result information 201. The interpretation report analysis unit 11 then collates each word of the word decomposition result information 201 with the structuralization dictionary 111 to specify which word corresponds to which class, for example, “region”, “symptom”, or “name of disease”. The interpretation report analysis unit 11 collates each word whose class is specified with a medical thesaurus dictionary to specify the meaning of each word, and generates analysis result information 202 including itemized medical meaning information. In this manner, in step S2, the interpretation report information created by the doctor or the like as a user is analyzed to generate analysis result information having medical information extracted from the interpretation report information associated with each item.

(Step S3)

The medical examination data input unit 12 inputs medical examination data including medical image data, that is, images to be interpreted (*1).

(Step S4)

The diagnosis support information generating unit 13 generates information of medical diagnosis suspected on an object based on the medical examination data input in step S3. That is, diagnosis support information is generated by computer processing of medical examination data.

CAD Which Handles Only Medical Image Data

If the medical examination data input in step S3 includes medical image data, the diagnosis support information generating unit 13 performs the image processing of detecting a lesion candidate based on image features in the medical image data. The diagnosis support information generating unit 13 then performs the discrimination processing of discriminating the authenticity or type of the detected lesion candidate, and generates, based on the result, diagnosis support information for the medical examination data. Note that the processing of generating diagnosis support information from medical examination data including medical image data is known, and hence a detailed description of the processing will be omitted.

CAD Which Also Handles Information Other Than Medical Image Data

The processing targets of the diagnosis support information generating unit 13 are not limited to medical image data and include, for example, past interpretation reports and medical records on the object and medical examination data including other information which can be used for diagnosis support processing. In this case, it is possible to generate medical diagnosis support information based on medical examination data other than the image information of the object. Note that the processing of generating diagnosis support information from medical examination data including medical image data is known, and hence a detailed description of the processing will be omitted.

(Step S5)

The difference detection unit 14 detects the difference between the analysis result information 202 extracted and generated from the interpretation report information, which is the interpretation result obtained by the doctor in step S2, and the diagnosis support information generated in step S4.

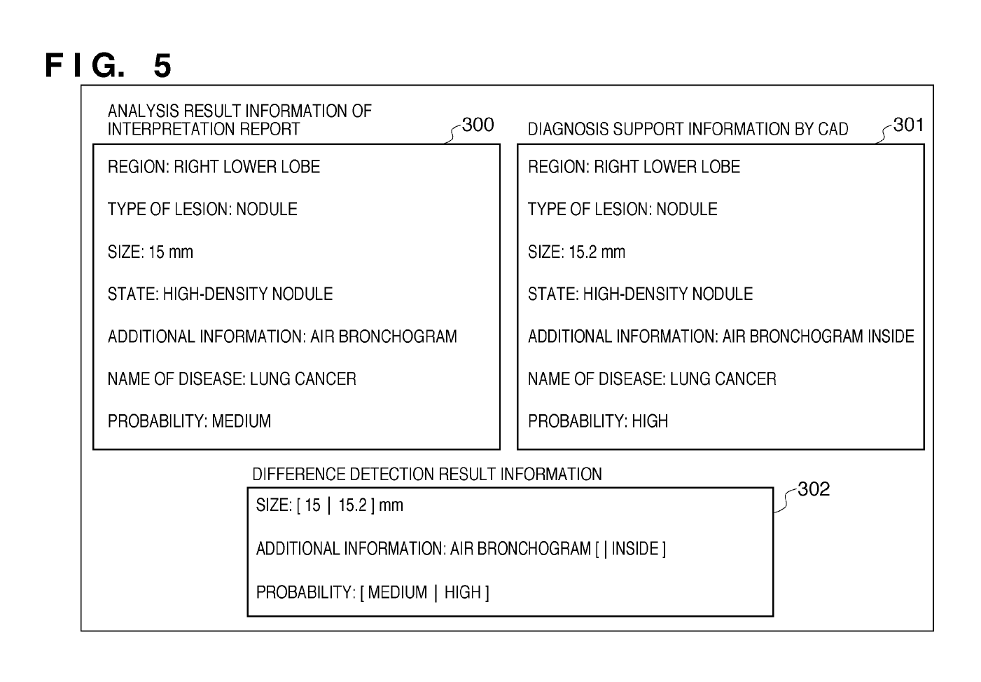

A concrete example of the processing in step S5 will be described with reference to FIG. 5. Analysis result information 300 of an interpretation report in FIG. 5 corresponds to the analysis result information 202 which is generated by the interpretation report analysis unit 11 in step S2 and represents the medical meaning of the interpretation report. Diagnosis support information 301 is the diagnosis support information generated by the diagnosis support information generating unit 13 in step S4. The analysis result information 300 of the interpretation report and the diagnosis support information 301 each are formed from medically significant itemized information. The difference detection unit 14 detects the presence/absence of a difference for each item and the contents of the difference, as indicated by difference detection result information 302, by comparing these pieces of information for each item. In this manner, a difference is detected for each item by comparing diagnosis support information, in which the medical information obtained by computer processing of medical examination data is associated with each item, with the analysis result information obtained by analysis on the interpretation report information.

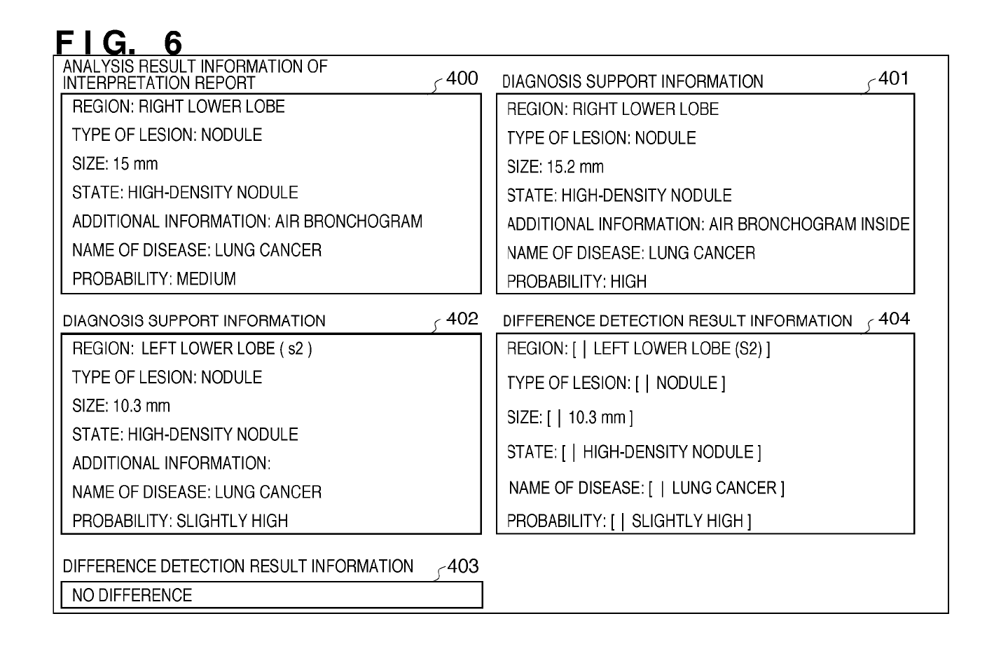

A concrete example of the processing in step S5 will be described with reference FIG. 6 in a case in which the information of the interpretation result obtained by the doctor, extracted in step S2, and/or the diagnosis support information generated in step S4 includes a plurality of pieces of finding information.

Analysis result information 400 of an interpretation report in FIG. 6 corresponds to the analysis result information 202 of the interpretation report generated by the interpretation report analysis unit 11 in step S2. Diagnosis support information 401 and diagnosis support information 402 are the plurality of pieces of diagnosis support information concerning different regions, which are generated by the diagnosis support information generating unit 13 in step S4.

In detecting differences, it is checked whether the diagnosis support information 401 and the diagnosis support information 402 include any findings corresponding to the analysis result information 400 of the interpretation report. A method of checking the correspondence between findings based on “region” information as an example will be described below. In the analysis result information 400 of the interpretation report, “right lower lobe” is extracted as the information of “region”. On the other hand, in the diagnosis support information 401 and the diagnosis support information 402, “right lower lobe (S1)” and “left lower lobe (S2)” are obtained. It is determined from these pieces of region information that the analysis result information 400 of the interpretation report corresponds to the diagnosis support information 401 including the information of the item “region” which is common to the analysis result information 400. The difference detection unit 14 obtains difference detection result information 403 by comparing the pieces of associated finding information (the analysis result information 400 and the diagnosis support information 401 in this case) by the same method as described above. The difference detection result information 403 indicates a case in which the detection result indicates no difference between the pieces of finding information. With regard to the diagnosis support information 402 having no correspondence with the analysis result information 400 of the interpretation report, since all the pieces of information of the diagnosis support information 402 are difference information, pieces of difference information including all the pieces of information of the diagnosis support information 402 as differences are detected, as indicated by difference detection result information 404. Note that in contrast to the size “15 mm” in the analysis result information 400, the size in the diagnosis support information 401 is “15.3 mm”. Although the accuracy (the number of digits) of a numerical value calculated by the computer differs from the accuracy (the number of digits) of a numerical value written by the doctor who has interpreted radiograms, since the difference detection unit 14 determines that both the numerical values indicate the same meaning, it is determined that there is no difference. As described above, it suffices to detect differences while accommodating differences in accuracy.

(Step S6)

The medical importance calculating unit 15 discriminates the presence/absence of a difference detected in step S5. If there is a difference, the process advances to step S7. If there is no difference, this processing is terminated.

(Step S7)

The medical importance calculating unit 15 calculates and determines a medical importance concerning difference detection information indicating the difference between the analysis result information of the interpretation result obtained by the doctor, detected in step S5, and the diagnosis support information. That is, the medical importance calculating unit 15 determines an importance concerning the difference for each item detected in step S5.

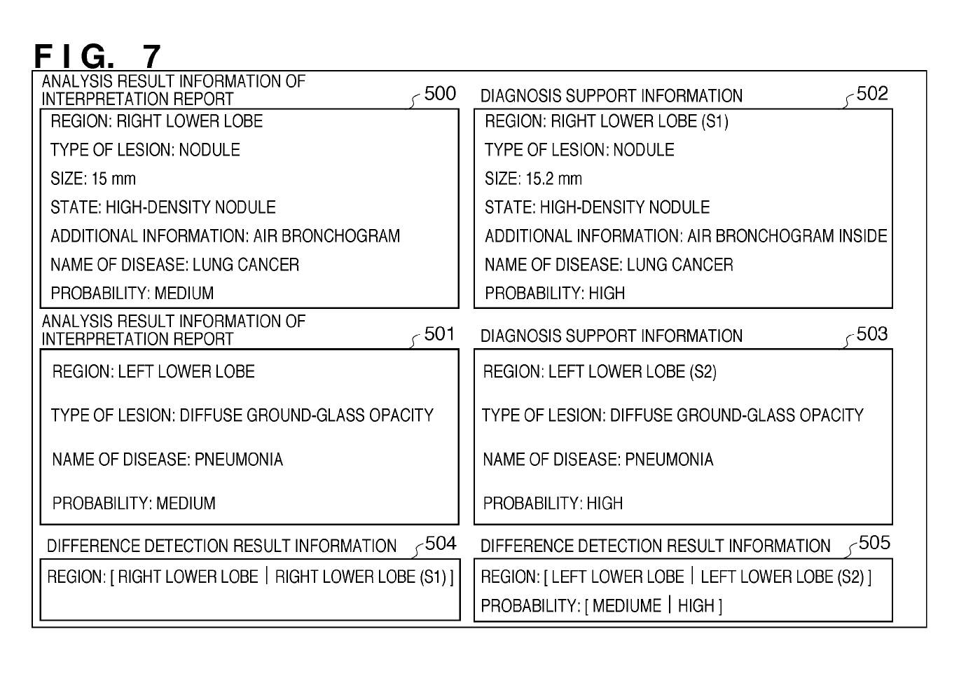

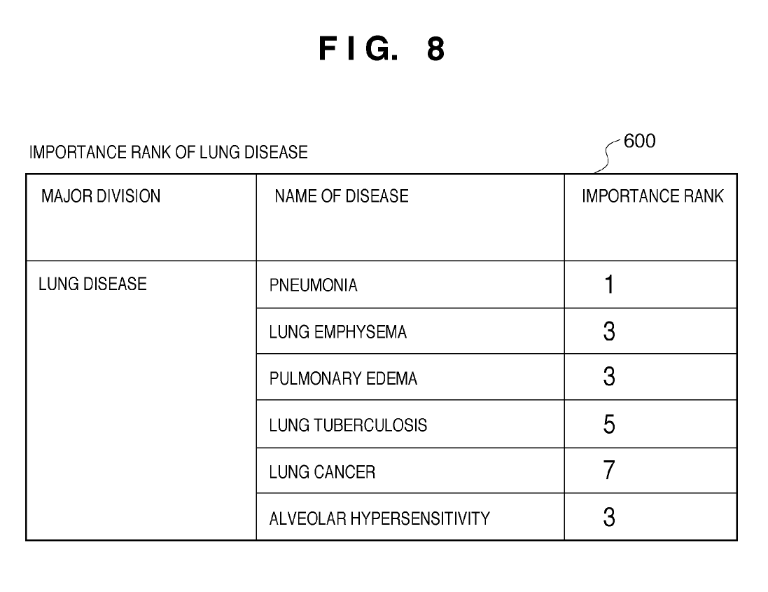

Concrete processing contents in step S7 will be described in detail with reference to FIGS. 7 and 8. Various techniques are conceivable for the calculation of a medical importance in step S7. This embodiment will exemplify a case in which an importance is determined by using importance ranks, each assigned to the name of a lesion in advance in accordance with a relationship with the maintenance of life of the object.

Consider a case in which pieces of analysis result information 500 and 501 of the interpretation report are obtained in step S2, and pieces of diagnosis support information 502 and 503 are obtained in step S4, as shown in FIG. 7. Assume also that in step S5, the difference detection unit 14
has determined based on region information that the analysis result information 500 and the diagnosis support information 502 correspond to the first corresponding findings, and the analysis result information 501 and the diagnosis support information 503 are the second corresponding findings, and
has obtained difference detection result information 504 of the first corresponding findings, and difference detection result information 505 of the second corresponding findings.

The term importance dictionary 150 forming the medical importance calculating unit 15 which performs the processing in step S7 has, for example, importance rank information 600 like that shown in FIG. 8 as a database in advance. Since the difference detection result information 504 shown in FIG. 7 is the difference detection result information between the analysis result information 500 and the diagnosis support information 502, the difference detection result information associates “lesion” with “lung cancer”. The medical importance calculating unit 15 refers to the importance rank information 600 to determine that the importance rank of “lung cancer” is “7”, and sets the importance rank of the difference detection result information 504 in FIG. 7 to “7”. On the other hand, the difference detection result information 505 in FIG. 7 is the difference detection result information between the analysis result information 501 and the diagnosis support information 503, and hence the difference detection result information associates “lesion” with “pneumonia”. The medical importance calculating unit 15 refers to the importance rank information 600 to determine that the importance rank of “pneumonia” is “1”, and sets the importance rank of the difference detection result information 505 in FIG. 7 to “1”.

(Step S8)

The difference presenting unit 16 presents the difference information detected in step S5 on the interpretation result obtained by the doctor, input in step S1, while changing the display in accordance with the medical importance calculated in step S7. In this case, for example, the difference presenting unit 16 can be configured to perform the processing of displaying the difference information detected in step S5 on the interpretation result obtained by the doctor, input in step S1, only when the medical importance calculated in step S7 is larger than a predetermined threshold. This processing will be described more specifically with reference to FIGS. 7 to 9.

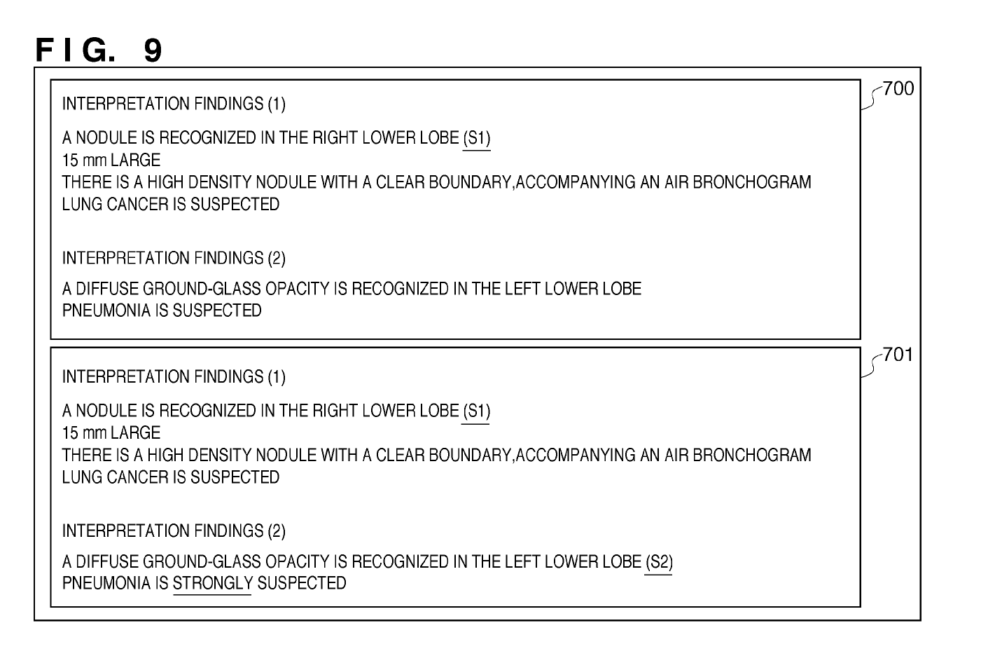

Consider first processing performed based on the medical importance calculated in step S7. As described with reference to step S7, the importance ranks of the difference detection result information 504 and difference detection result information 505 in FIG. 7 have been calculated as 7 and 1, respectively. In this case, if, for example, the medical importance rank threshold is set to 5, the difference presenting unit 16 displays only differences of finding, which are associated with a lung cancer whose medical importance rank exceeds 5, together with the interpretation result input in step S1, as indicated by presented contents 700 in FIG. 9. If the medical importance rank threshold is set to 0, the difference presenting unit 16 displays all the pieces of difference information together with the interpretation result input in step S1, as indicated by presented contents 701 in FIG. 9.

As described above, according to the arrangement of the first embodiment, it is possible to present, on an interpretation report, some of differences between the interpretation report written by the doctor and the diagnosis support information calculated by the computer which are medically important. This makes it possible to present medically important difference information without bothering the doctor due to the presentation of medically unimportant difference information.

### Second Embodiment

The first embodiment has exemplified, as concrete processing in the medical importance calculating unit 15, the processing of defining medical importances in accordance with the contents of detected difference information. However, the processing performed by the medical importance calculating unit 15 of the present invention is not limited to this. The following will describe other several embodiments of the medical importance calculating unit 15.

<Calculation of Medical Importance Based on Class to Which Information Belongs>

The medical importance calculating unit 15 can define the difference result information detected by a difference detection unit 14 in accordance with the class to which the difference information belongs. For example, the class to which a word appearing in difference result information belongs is classified to one of “region”, “region supplementation”, “symptom”, “symptom supplementation”, “name of disease”, and “examination/treatment”. Higher medical importances are assigned to differences concerning “region”, “symptom”, and “name of disease”, and low medical importances are assigned to differences concerning “region supplementation”, “symptom supplementation”, and “examination/treatment”. This makes it possible to calculate a medical importance for the difference information detected by the difference detection unit 14 in accordance with the term class to which the difference information belongs.

<Calculation of Medical Importance Based on Magnitude of Difference Between Contents>

The medical importance calculating unit 15 may calculate a medical importance in accordance with the magnitude of the difference detected by the difference detection unit 14. If, for example, a difference in the size of a pulmonary nodule is detected as a difference belonging to the class “symptom supplementation”, the medical importance calculating unit 15 can calculate a medical importance in accordance with a difference in numerical information indicating the size. In this case, for example, the difference in numerical value between “1 mm” and “2 mm” can be set to “difference: 1 mm” as an absolute value difference. In addition, “difference: double” can be calculated as the ratio between the magnitudes of numerical values, and a medical importance corresponding to the ratio can be calculated.

It is also possible to calculate a medical importance in accordance with the magnitude of a semantic difference which text information such as word information has instead of a difference in numerical information. In this case, for example, “lung emphysema” and “chronic bronchitis” are both kinds of obstructive pulmonary diseases, and hence the medical importance of the difference is calculated as a small value. On the other hand, “lung emphysema” and “lung cancer” are different kinds of legions, and hence the medical importance of the difference can be calculated as a large value. This makes it possible to calculate a medical importance in accordance with the magnitude of a medically semantic difference. Assume that the magnitudes of “differences” between such names of diseases are registered as a table in advance.

<Calculation of Medical Importance Based on Combination of Difference Contents>

The medical importance calculating unit 15 can also calculate a medical importance in accordance with a combination of difference contents detected by the difference detection unit 14. Assume that the name of a serious lesion is written as diagnosis support information in spite of the fact that the name of a disease having a slight influence on the maintenance of life of an object is written on the interpretation report written by the doctor. In this case, the medical importance can be increased. In addition, if the names of non-serious lesions are written on both the interpretation report written by the doctor who has interpreted radiograms and the diagnosis support information generated by the computer, even though they have a difference, the medical importance can be decreased.

<Calculation of Medical Importance Based on Presence/Absence of Corresponding Finding>

The medical importance calculating unit 15 can also calculate a medical importance, based on the detection result obtained by the difference detection unit 14, in accordance with whether there is an interpretation result corresponding to diagnosis support information on the interpretation report. If, for example, there is no interpretation result corresponding to diagnosis support information, a predetermined importance is assigned to the diagnosis support information. Alternatively, an importance may be set in advance in accordance with a class (item) when there is no interpretation result. If there is no interpretation result corresponding to diagnosis support information, the above set importance is assigned to the word in accordance with the class (item) to which the word belongs. In this case, for example, increasing a medical importance for diagnosis support information having no correspondence with an interpretation report can prevent the doctor from making an oversight.

<Others>

The medical importance calculating unit 15 can be configured to calculate medical importances by a combination of various calculation methods associated with the above calculation of medical importances. For example, it is possible to calculate a medical importance in consideration of both a medical importance calculated in accordance with the class to which a word of a portion corresponding to a difference detected as a result of difference detection belongs and another medical importance calculated in accordance with the magnitude of the difference between contents written on the difference portions.

### Third Embodiment

The first embodiment has exemplified the processing of switching whether to present difference information in accordance with the medical importance, as concrete processing in the difference presenting unit 16. However, the processing in the difference presenting unit 16 of the present invention is not limited to this. Another embodiment of the difference presenting unit 16 will be described below.

<Types of Expression>

A difference presenting unit 16 can present the difference information detected by a difference detection unit 14 on the interpretation report input by an interpretation report input unit 10 while switching the display form in accordance with the medical importance calculated by the medical importance calculating unit 15. For example, the difference presenting unit 16 can present difference information while switching the display forms of characters, for example, the sizes of characters, the types of fonts, the colors of characters, the blinking states of characters, and the use/non-use of popup display, in accordance with a medical importance. The display form to be changed is not limited to the display form of characters itself. It is possible to use other display forms such as the background colors of characters and annotation display that prompts popup. This allows the user to recognize the importance of difference information in accordance with differences in display form of characters and others.

<Case of No Difference>

The difference presenting unit 16 can also be configured to present “no difference” or characters or symbols which have meanings similar thereto, even when no difference is presented, based on the processing results obtained by the difference detection unit 14 and the medical importance calculating unit 15. With this arrangement, even when no difference is presented, the user can discriminate whether no difference has been presented as a result of difference detection processing or the system itself has not operated normally.

### Other Embodiments

Although an embodiment has been described in detail above, the present invention can take embodiments as a system, apparatus, method, program, storage medium, and the like. The present invention can be applied to a system including a plurality of devices, or to an apparatus including a single device.

The present invention incorporates a case in which programs of software are directly or remotely supplied to a system or apparatus to cause the computer of the system or apparatus to read out and execute the program codes, thereby implementing the functions of the above embodiments. In this case, the supplied programs are computer programs corresponding to the flowcharts shown in the accompanying drawings in the embodiments.

The program codes themselves which are installed in the computer to allow the computer to implement the functions/processing of the present invention also implement the present invention. That is, the present invention incorporates the computer programs themselves for implementing the functions/processing of the present invention.

In this case, each program may take any form, for example, an object code, a program executed by an interpreter, and script data supplied to an OS, as long as it has the function of the program.

A computer-readable storage medium for supplying the computer programs includes, for example, a floppy (registered trademark) disk, hard disk, optical disk, magnetooptical disk, MO, CD-ROM, CD-R, CD-RW, magnetic tape, nonvolatile memory card, ROM, DVD (DVD-ROM or DVD-R), or the like.

In addition, methods of supplying the programs include the following. A client computer connects to a homepage on the Internet by using a browser to download each computer program of the present invention from the homepage into a recording medium such as a hard disk. In this case, the program to be downloaded may be a compressed file containing an automatic install function. Alternatively, the programs can be supplied by dividing the program codes constituting each program of the present invention into a plurality of files, and downloading the respective files from different homepages. That is, the present invention also incorporates a WWW server which allows a plurality of users to download program files for causing the computer to execute the functions/processing of the present invention.

In addition, the functions/processing of the present invention can be implemented by encrypting the programs of the present invention, storing the encrypted data in storage media such as CD-ROMs, and distributing them to users. In this case, users who satisfy a predetermined condition are allowed to download key information for decryption from a homepage through the Internet. Executing the encrypted programs using the key information allows a computer to install the programs.

The functions of the above embodiments are implemented by making the computer execute the readout programs. In addition, the functions of the above embodiments can also be implemented by making the computer operate in cooperation with the OS or the like running on the computer based on the instructions of the programs. In this case, the OS or the like performs part or all of actual processing to implement the functions of the above embodiments.

Part or all of the functions of the above embodiments are also implemented by writing the programs read out from the recording medium in the memory of a function expansion board inserted into the computer or a function expansion unit connected to the computer. In this case, after the programs are written in the function expansion board or the function expansion unit, the CPU or the like of the function expansion board or function expansion unit performs part or all of actual processing based on the instructions of the programs.

The present invention is not limited to the above embodiments and various changes and modifications can be made within the spirit and scope of the present invention. Therefore, to apprise the public of the scope of the present invention, the following claims are made.

This application claims the benefit of Japanese Patent Application No. 2007-252378, filed Sep. 27, 2007 which is hereby incorporated by reference herein in its entirety.

1. A medical diagnosis support apparatus which supports diagnosis by a doctor based on medical examination data, said apparatus comprising:
an analysis unit configured to analyze interpretation report information created by a user and generate analysis result information in which each medical information extracted from the interpretation report information is associated with an item;
a detection unit configured to detect a difference for each item by comparing diagnosis support information, in which each medical information obtained by computer processing of medical examination data is associated with an item, with the analysis result information obtained by said analysis unit;
a determination unit configured to determine an importance concerning a difference for each item detected by said detection unit; and
a presentation unit configured to present the difference detected by said detection unit, together with display of contents of the interpretation report information, while changing a display form in accordance with the importance determined by said determination unit,
wherein said determination unit determines a magnitude of the difference detected by said detection unit and determines an importance of the difference based on the determined magnitude of the difference.

2. The medical diagnosis support apparatus according to claim 1, wherein said presentation unit presents only a difference having an importance higher than a set threshold, together with the contents of the interpretation report information.

3. The medical diagnosis support apparatus according to claim 1, wherein said presentation unit presents the difference while changing a display form of characters in accordance with the importance determined by said determination unit.

4. The medical diagnosis support apparatus according to claim 3, wherein the display form of characters which said presentation unit changes includes at least one of a size of character, a font, a color of a character, a background color of a character, and a form of popup display.

5. The medical diagnosis support apparatus according to claim 1, wherein said determination unit determines an importance of the difference based on a name of a disease indicated by the analysis result information from which the difference is detected by said detection unit.

6. The medical diagnosis support apparatus according to claim 1, wherein said determination unit determines an importance of the difference based on a type of item to which the difference detected by said detection unit corresponds.

7. (canceled)

8. The medical diagnosis support apparatus according to claim 1, wherein said presentation unit performs display indicating that there is no difference, when no difference is detected by said detection unit.

9. (canceled)

10. A control method for a medical diagnosis support apparatus which supports diagnosis by a doctor based on medical examination data, said control method comprising:
an analysis step of analyzing interpretation report information created by a user and generating analysis result information in which each medical information extracted from the interpretation report information is associated with an item;
a detection step of detecting a difference for each item by comparing diagnosis support information, in which each medical information obtained by computer processing of medical examination data is associated with an item, with the analysis result information obtained in the analysis step;
a determination step of determining an importance concerning a difference for each item detected in the detection step; and
a presentation step of presenting the difference detected in the detection step, together with display of contents of the interpretation report information, while changing a display form in accordance with the importance determined in the determination step,
wherein in the determination step, a magnitude of the difference detected in the detection step is determined and an importance of the difference based on the determined magnitude of the difference is determined.

11. (canceled)

12. A computer readable medium storing thereon a computer program for causing a computer to execute medical diagnosis support to support diagnosis by a doctor based on medical examination data, the computer program causing the computer to execute:
an analysis step of analyzing interpretation report information created by a user and generating analysis result information in which each medical information extracted from the interpretation report information is associated with an item;
a detection step of detecting a difference for each item by comparing diagnosis support information, in which each medical information obtained by computer processing of medical examination data is associated with an item, with the analysis result information obtained in the analysis step;
a determination step of determining an importance concerning a difference for each item detected in the detection step, wherein a magnitude of the difference detected in the detection step is determined and an importance of the difference based on the determined magnitude of the difference is determined; and
a presentation step of presenting the difference detected in the detection step, together with display of contents of the interpretation report information, while changing a display form in accordance with the importance determined in the determination step.

13-14. (canceled)
